SEQUENCE LISTING (3 sequences)

SEQ ID NO: 1 - DNA, 33 bp, Artificial Sequence
  Oligonucleotode
tccaagcctggagggatgatactttgcgcttgg

SEQ ID NO: 2 - DNA, 33 bp, Artificial Sequence
  Oligonucleotode
ttggaatccatggttaaacttgtactttaggtc

SEQ ID NO: 3 - DNA, 32 bp, Artificial Sequence
  Oligonucleotode
ccaagcgcaaagtatcatccctccaggcttgg

(19) United States
(12) Patent Application Publication
(10) Pub. No.: US 2003/0186257 A1
(43) Pub. Date: Oct. 2, 2003

(54) Method for identifying a mark applied on a solid body

(76) Inventors: Wolf Bertling, Erlangen (DE); Hans Kosak, Bonn (DE); Andr?eacute; Josten, Hemhofen (DE); Georg Bauer, N?uuml;rnberg (DE); Harald Walter, Erlangen (DE)

Correspondence Address: Fish & Richardson, Suite 3300, 60 South Sixth Street, Minneapolis, MN, 55402, US

(21) Appl. No.: 10/169,919

(22) PCT Filed: Jan. 9, 2001
(86) PCT No.: PCT/DE01/00055

(30) Foreign Application Priority Data
Jan. 10, 2000 (DE) 100-00-629..9

(51) Int. Cl. C12Q 1/68

(52) U.S. Cl. 435/6

(57) ABSTRACT

The invention relates to a method for identify a predetermined mark (32) applied on a solid body (30) and constituted by planar elements (10). The inventive method comprises the following steps: (a) binding first biopolymers to a first part of the planar elements (10) so as to produce a first predetermined partial pattern, (b) contacting the mark (32) with third biopolymers that have an affinity to the first biopolymers so that the first and the third biopolymers bind to one another, and (c) identifying the first partial pattern produced by the bound first and third biopolymers by detecting the bond between the first and the third bipolymers by means of a one-stop detection method.

The invention relates to a method for identifying a mark applied on a solid body and formed from area elements, to a carrier and to a kit.

The invention relates in particular to the area of security, coding and identification technology.

DE 197 38 816 A1 discloses the extraction or removal from the solid of nucleic acids bound to a solid for marking. The nucleic acids undergo dissolution. They are multiplied by a specific reaction such as PCR. The multiplied nucleic acid sequence is then analyzed. The method is time-consuming. Extraction of the nucleic acid applied for marking is not possible or desired with every solid.

A method for identifying a mark provided on a solid is disclosed in DE 198 11 730 A1. The mark in this case has a nucleotide sequence. The nucleotide sequence is brought into contact with a corresponding nucleotide sequence which is bound to a solid phase of a detection means. For satisfactory hybridization, the solid phase of the detection means must be pressed against the mark. This makes the identification difficult.

U.S. Pat. No. 5,139,812 discloses the use of a predetermined nucleic acid-containing ink for forgeryproof marking of articles. For distinguishable marking of a plurality of articles, different inscriptions are applied with the ink. A mark applied in this way is identified by binding another nucleic acid to the predetermined nucleic acid. The bound nucleic acid can be visualized by a color reaction or on the basis of a radiolabel. The mark can be revealed by a sequence-nonspecific nucleic acid binding without knowledge of the sequence used for marking. The method is not secure.

EP 0 745 690 A2 describes so-called molecular beacons and the use thereof for hybridization. A use for detecting marks is not disclosed in this document.

U.S. Pat. No. 5,866,336 describes primers labeled with a fluorophore. The primers are hybridized by polymerase chain reaction. In the hybridized state, refolding of the primers is broken up. The fluorescence behavior of the fluorophore provided on the primer is thus altered. The known method is unsuitable for rapid identification of a mark because it requires the cost-intensive and time-consuming polymerase chain reaction.

DE 199 01 761 discloses a method for detecting the hybridization of DNA by means of a change in a redox potential. Such a change in the redox potential cannot be measured straightforwardly. The known method does not permit rapid and simple identification of a mark.

It is an object of the present invention to eliminate the disadvantages of the prior art. It is intended in particular to indicate an alternative method with which a reliable identification of a mark applied on a solid body is possible rapidly and simply.

The object is achieved by the features of claims 1 and 24. Expedient developments of the invention are evident from the features of claims 2 to 23 and 25 to 29.

The invention provides a method for identifying a predetermined mark applied on a solid body and formed from area elements, having the following steps:

a) binding of first biopolymers to a first part of the area elements so that a first part-pattern is formed.

b) bringing the mark into contact with third biopolymers having affinity for the first biopolymers, so that the first and the third biopolymers bind to one another and

c) identifying the first part-pattern formed by the bound first and third biopolymers through detecting the bindings between the first and third biopolymers by means of a one-stage detection method.

The biopolymers may be bound covalently or noncovalently to the area elements. They may also be synthesized directly on the area elements. Biopolymers have affinity for other biopolymers when they are able to bind specifically to these.

The method of the invention makes reliable identification of a mark applied on a solid body possible. It is possible in particular for the mark to be identified directly on the product without needing to be detached therefrom.

In an advantageous development, the following step is carried out before step b: binding of second biopolymers to a second part of the area elements so that a second part-pattern is formed. The area elements with second biopolymers bound thereto prevent nonspecific identification of area elements with first biopolymers bound thereto. Under appropriate conditions it is possible for nonspecific third biopolymers to bind to area elements with first biopolymers bound thereto. However, they also bind to all other area elements and do not make identification of the mark possible. Such an identification is possible only if the relevant specific third biopolymers are known. This makes the method very secure. The method can additionally be carried out rapidly and simply.

The biopolymers may comprise, in particular synthetic and/or single-stranded, nucleic acids, analogs thereof, antigens or proteins, in particular antibodies, antibody fragments, derivatives of antibodies or antibody fragments or nucleic acid-binding proteins. Protein-protein, nucleic acid-nucleic acid or nucleic acid-protein interactions may occur between the biopolymers on binding. It is moreover possible for the nucleic acid also to be replaced in each case by a nucleic acid analog. Protein-protein interactions may occur between antibodies and antigens. Antigens comprise every molecule which can be bound specifically by an antibody, an antibody fragment or a derivative of an antibody or antibody fragment. The antigen may be produced purely synthetically. It need not be a derivative of a biological molecule.

In an advantageous development, in step b) additionally fourth biopolymers having affinity for the second biopolymers are brought into contact with the mark. In step c) the bindings between the second and the fourth biopolymers are detected and the part-patterns formed by the bound second and fourth biopolymers are identified. Detection of the second biopolymers is possible only if the relevant specific fourth biopolymers are known. Such a method is more secure than a method in which only a first biopolymer is specifically identified. A clear contrast can be produced between the part-pattern formed by the bound first and third biopolymers and the part-pattern formed by the bound second and fourth biopolymers. It is possible for this purpose to provide the third and fourth biopolymers with clearly distinguishable marking substances. The sharpness of separation between the part-patterns is distinctly greater than on detection only of the part-pattern formed by the bound first and third biopolymers. This is particularly advantageous when the part-pattern is very small or narrow. The third and, where appropriate, the fourth biopolymers may be present in a solution. This ensures simple manipulation of the method.

In a further advantageous development, the bringing into contact is carried out under predetermined stringent binding conditions, preferably at room temperature. Stringent binding conditions are conditions under which the third and, where appropriate, the fourth biopolymers bind essentially only to those biopolymers with which they have affinity. Nonspecific binding to other biopolymers essentially do [sic] not take place. Stringent binding conditions can be achieved by appropriate temperature or ionic strength. In the case of nucleic acids as biopolymers, the stringent binding conditions can be determined by the choice of appropriate nucleotide sequences. Adaptation of the stringent binding conditions to the particular purpose of the marking is thus possible. It is advantageous if the nucleic acids differ as widely as possible in their nucleotide sequences. Nonspecific hybridizations are thus very unlikely.

It is advantageous for at least one other or the second biopolymer to be bound to the area elements to saturate nonspecific binding sites. This prevents nonspecific binding of the third and/or fourth biopolymer to the background in the region of the area elements. It is unnecessary to block the nonspecific binding sites on the area elements directly before identifying the mark. This makes the method inter alia very rapid.

In one development, the first and second biopolymer are bound via hydrophilic linkers respectively to the one or other part of the area elements. The hydrophilic linkers can be selected from the following group: peptides, polyethylene glycols, polymeric sugars, polyacrylamide, polyimines, dendrimer molecules. The provision of such linkers improves the accessibility of the biopolymers for the third and, where appropriate, the fourth biopolymers. It is additionally expedient for the hydrophilic linker to be bound to the first or third biopolymer in a section which is not complementary respectively to the second or fourth biopolymer. This ensures hybridization of the biopolymers which are complementary with one another. The linker may advantageously also be bound terminally to the first or third biopolymer. The sensitivity of the method is increased. In addition, at least one of the biopolymers can be bound to the area elements by means of particles, in particular agarose particles. This is advantageous especially when the surface of the solid body does not allow the biopolymers or linkers to be bound directly thereto.

It is particularly advantageous for at least one of the biopolymers to be applied by means of a printing technique, in particular inkjet technique, to the area elements. Such a method makes it possible for different marks to be applied in a large number automatically to solid bodies, e.g. packages, in a production run.

In a further development, the first and/or second biopolymers are bound at a predetermined site in their structure to the area elements. It is possible by this measure to prevent the first and/or second biopolymers binding at their binding sites for the third and fourth biopolymers to the area elements. For example, in the case of antibodies, it is important that they bind with their F, parts and not with their antigen-binding sites to the area elements. A defined binding can be achieved by coating the area elements with protein A or with protein G. These proteins specifically bind the F_{c} parts of the antibodies brought into contact therewith.

The part-pattern may be in the form of a bar code. It is advantageous for the part-pattern to be designed in the form of an array. The area elements may be designed to be round, preferably with a diameter of less than 100 μm.

In a further development, the binding is detected through altered optical and/or electrical properties of the bound biopolymers. One optical property is, for example, the absorption capacity for light of particular wavelengths. The alteration in the absorption capacity due to the binding may lead to a change in color. An electrical property is, for example, the conductivity. Detection through altered properties requires neither chemical nor biochemical detection reaction. Extraction or removal of the bound biopolymers from the solid body is not necessary. The identification takes place simply and rapidly.

At least one of the biopolymers may have a fluorophore which changes its fluorescence properties on binding. Such a biopolymer may be designed for example in the form of a so-called molecular beacon disclosed in EP 0 745 690 A2. The binding of such a molecule to an appropriate complementary nucleic acid leads to a distinct enhancement of its fluorescence. The fluorescence can be detected immediately after the binding. Bound biopolymers can be recognized with the naked eye on suitable choice of the marking substance.

It is additionally possible for at least one of the biopolymers to have a marking substance which changes redox potential thereof on binding. The binding of such a biopolymer can be detected by means of an appropriate electrode.

In a preferred development, the third and/or fourth biopolymers are brought into contact with the mark homogeneously distributed by dropwise application, absorption, spraying or atomization. Such a method has the advantage of being very simple to manipulate. The third and/or fourth biopolymers can be sprayed in solution, e.g. from a spray can, onto the mark. Specifically bound biopolymers can be detected a short time later.

The one-stage detection method is expediently a method which is carried out without washing steps. The one-stage detection methods may moreover be carried out utilizing one of the following effects: formation or separation of a donor/acceptor pair, surface plasmon resonance, weight difference, inclusion or release of intercalators. It is particularly advantageous to utilize the formation or separation of a donor/acceptor pair. Such an effect occurs for example on use of molecular beacons.

The mark advantageously comprises the first biopolymer in an amount not exceeding 10 μg. The method requires extremely small amounts of biopolymers.

The object of the invention is further achieved by providing a carrier for attachment to a solid body, where a predetermined mark formed from area elements is applied to one side of the carrier, where first biopolymers are bound to a first part of the area elements so that a first part-pattern is formed, and where the carrier is designed as a sheet which is coated on one side with adhesive. Such a carrier can easily be attached to solid bodies to be marked.

In a further development, second biopolymers are bound to a second part of the area elements so that a second part-pattern is formed. This makes particularly reliable identification of the first part-pattern possible.

One side, i.e. the side coated with biomolecules, may be covered by a detachable protective sheet. It is likewise possible for the adhesive layer to be covered by another detachable protective sheet.

The invention further provides a kit comprising a carrier of the invention and comprising a third biopolymer having affinity for the first biopolymer. This biopolymer may be present in solution. The kit may further comprise a fourth biopolymer having affinity for the second biopolymer.

All suitable materials come under consideration for production of the carrier. Sheets produced from plastic or metal are particularly preferred.

The features which have been mentioned and those to be explained hereinafter can be used not only in the particular combinations indicated but also in other combinations or alone. Further advantages are evident from the following exemplary embodiments and in connection with the drawings. These show:

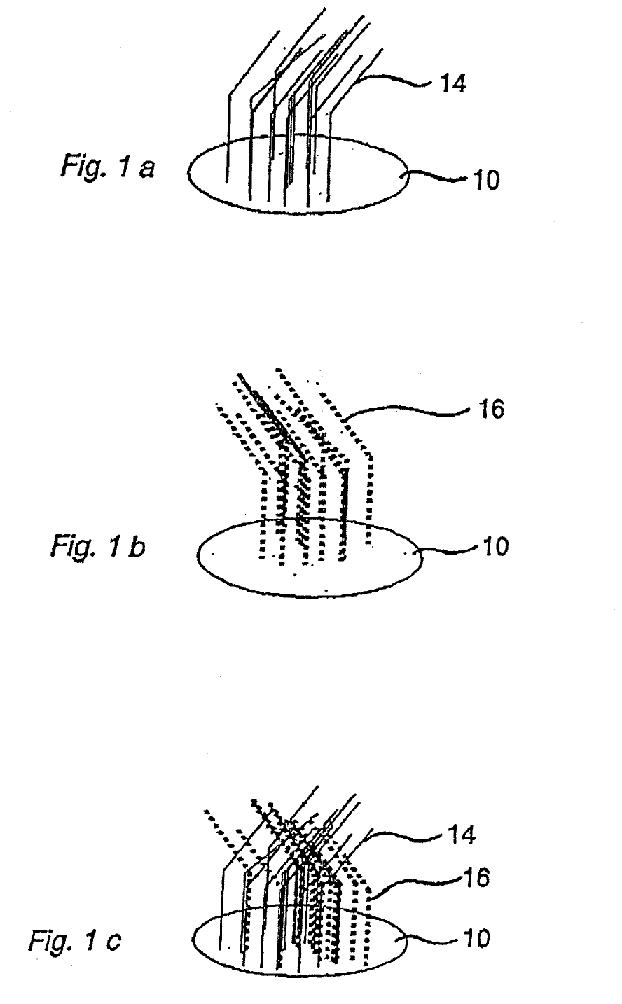

FIGS. 1a, b, c a diagrammatic representation of area elements with nucleic acids bound thereto,

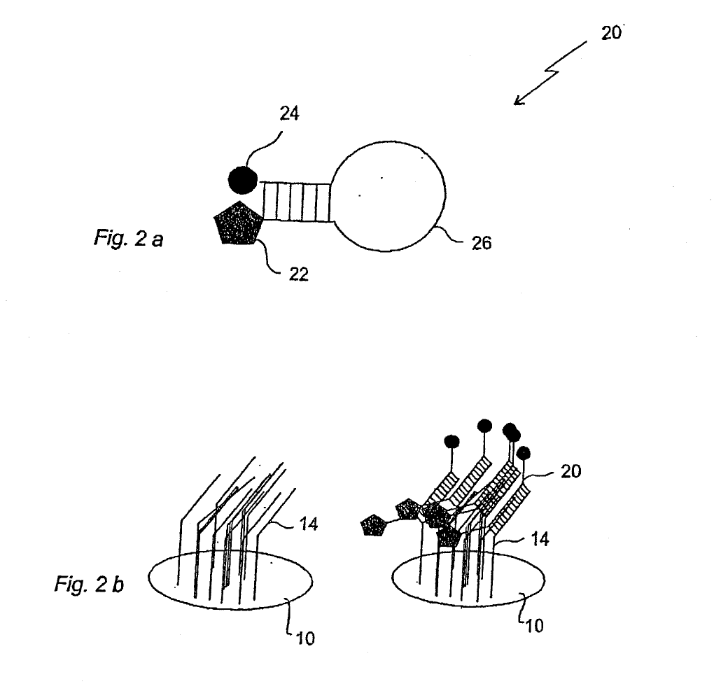

FIGS. 2a, b a diagrammatic representation of the identification of an area element with nucleic acids bound thereto by molecular beacons,

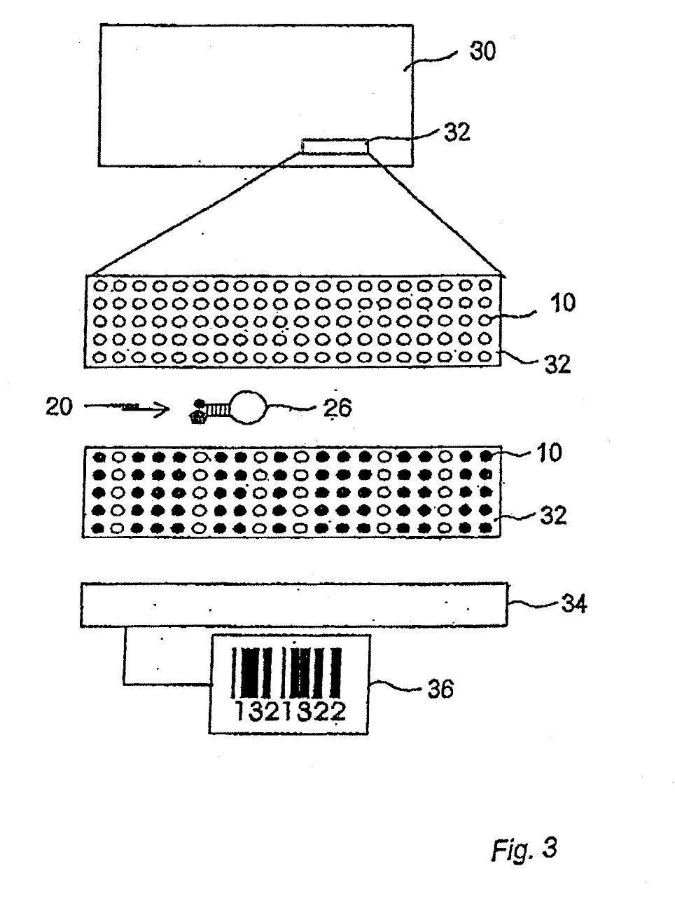

FIG. 3 a diagrammatic representation of the identification of a mark applied to a solid body,

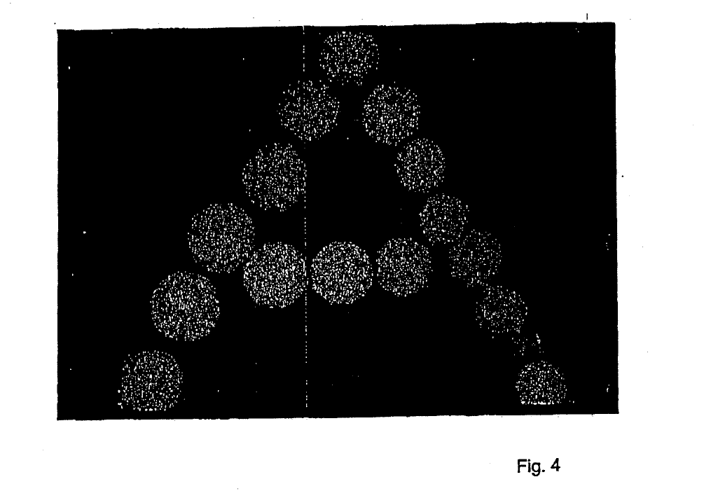

FIG. 4 a first part-pattern, consisting of particles, of a first exemplary embodiment in transmitted light,

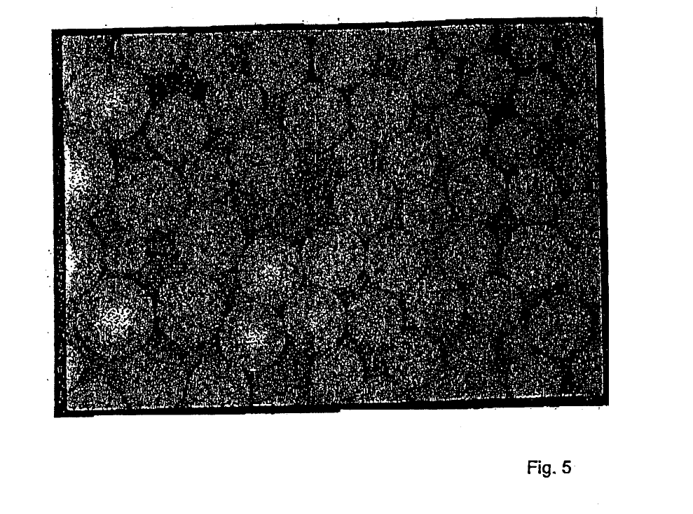

FIG. 5 the part-pattern shown in FIG. 4 together with a second part-pattern, formed from other particles, in transmitted light and

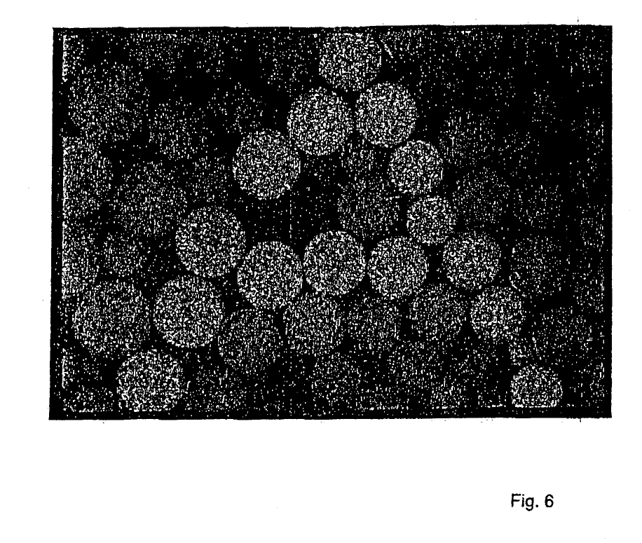

FIG. 6 the part-pattern shown in FIG. 5 with UV excitation.

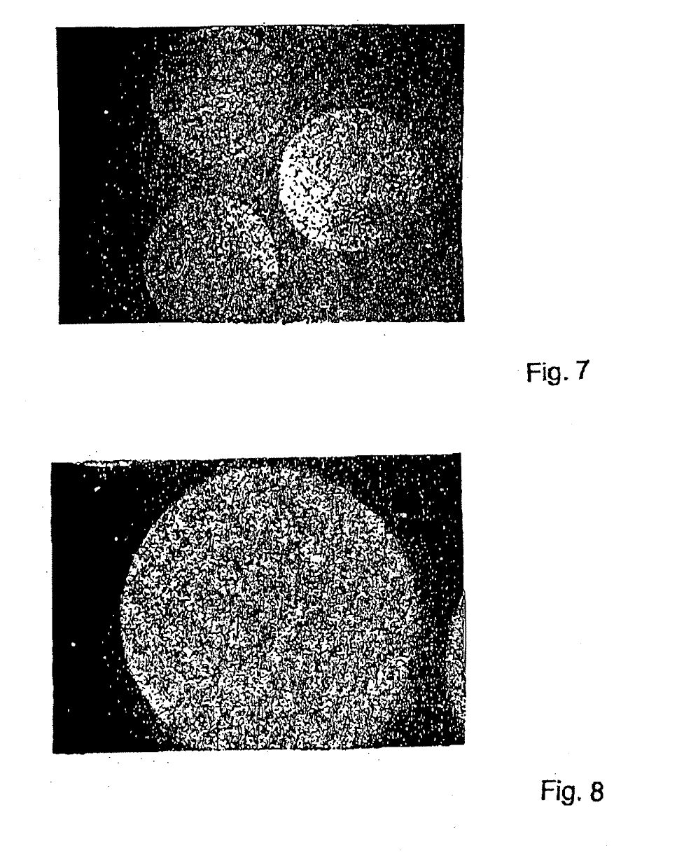

FIG. 7 a second exemplary embodiment,

FIG. 8 an enlarged representation of FIG. 7,

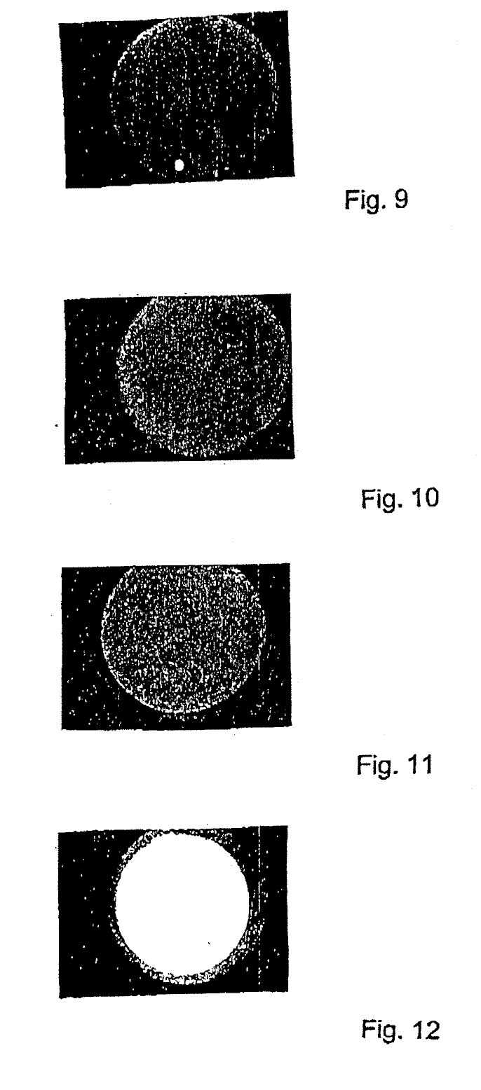

FIG. 9 a third exemplary embodiment produced with a concentration of 0.5 pmol/μl,

FIG. 10 the exemplary embodiment of FIG. 9 produced with a concentration of 1.0 pmol/μl,

FIG. 11 the exemplary embodiment of FIG. 9 produced with a concentration of 2.0 pmol/μl and

FIG. 12 the exemplary embodiment of FIG. 9 produced with an incubation time of 6 hours (concentration 2.0 pmol/μl).

FIG. 1a shows an area element 10 with first nucleic acids 14 bound thereto. FIG. 1b depicts an area element 10 with second nucleic acids 16 bound thereto. FIG. 1c shows an area element 10 with first 14 and second nucleic acids 16 bound thereto.

FIG. 2a is a diagrammatic representation of a molecular beacon 20. This takes the form of a hairpin-shaped DNA molecule. The DNA strand of this DNA molecule has regions complementary to one another at its ends. These regions are in base-paired form. At one end of the DNA strand there is a fluorophore 22, such as fluorescein, and at the other end there is a quencher 24, such as 4-dimethylaminoazobenzene-4′-sulfonyl chloride. When the molecular beacon 20 is irradiated with light of an excitation wavelength of the fluorophore 22 there is no emission of light. Instead there is a radiationless energy transfer to the quencher 24. In the loop 26 of the molecular beacon 20 there is a nucleotide sequence (not shown here) which is complementary to a nucleotide sequence of the first nucleic acid 14.

FIG. 2b shows on the left a diagrammatic representation of an area element 10 with first nucleic acids 14 bound thereto. On the right, this area element 10 is depicted after the binding of molecular beacons 20. The molecular beacons 20 bind with the nucleotide sequences in the loops 26 to the complementary nucleotide sequences of the first nucleic acids 14. This leads to breaking of the base pairings in the region of the ends of the DNA strands of the molecular beacons 20. The fluorophores 22 are spatially separated from the quenchers 24 by the binding. A radiationless energy transfer from the fluorophores 22 to the quenchers 24 is no longer possible. When the fluorophores 22 are excited with light of an excitation wavelength there is an emission of light which is measurable or even visible with the naked eye.

FIG. 3 shows a solid body 30, such as, for example, a banknote, with a mark 32. The mark 32 consists of an array of area elements 10. First nucleic acids 14, which are not depicted here, are bound to one part of the area elements 10. These each have a nucleotide sequence which is complementary to the nucleotide sequence of the loop 26 of a molecular beacon 20. Second nucleic acid sequences 16, which are likewise not shown here and which are not complementary thereto, are bound to the other part of the area elements. In addition, another nucleic acid is bound to the area elements 10 to saturate nonspecific binding sites. The molecular beacons 20 are present in a solution. The mark 32 is brought into contact with this solution. In order to ensure stringent binding conditions, the solution has a defined ionic strength, and the bringing into contact takes place at an elevated temperature. Under these conditions, the molecular beacons 20 bind via the first nucleic acid 14 only to one part of the area elements 10. They do not bind nonspecifically to the area elements 10 because nonspecific binding sites have been saturated. Nor do they bind to the other nucleic acids used for saturation or to the second nucleic acids 16 on the other part of the area elements 10.

If the stringency of the binding conditions were to be reduced, it would also be possible for nonspecific molecular beacons to bind to the first 14 and second nucleic acids 16. Identification of the part-pattern is impossible in this case.

Area elements 10 with bound molecular beacons 20 are depicted as circular areas filled with black, and the others are depicted as unfilled circular areas. On irradiation of the mark 32 with light of a suitable wavelength, the bound molecular beacons 20 fluoresce. A detector 34 measures and localizes the fluorescence. It represents the produced part-pattern on an output device 36. The security of the method can be increased by additionally detecting the second nucleic acids 16 which are bound to the other part of the area elements using specific other molecular beacons which are not depicted here. The other molecular beacons have a fluorophore different from the molecular beacons 20 and having a distinctly different fluorescence. This makes it possible to detect both specifically bound molecular beacons 20 and specifically bound other molecular beacons. The contrast between one part and the other part of the area elements is distinctly increased compared with the contrast on use only of the molecular beacons 20. The improved contrast increases the reliability on reading the fluorescence. This makes it possible to identify small or narrow part-patterns.

The mark shown in FIGS. 4 to 6 had been produced as follows:

Firstly slides made of glass are incubated successively for 30 minutes each in water, 6% ammonia, 5% H₂O₂, water, acetone and 2% 3-aminopropyltriethoxsilane [sic] in acetone, and then in acetone. The slides pretreated in this way are then dried at 37° C. for one hour.

The mark is produced by using preferably crosslinked 4% aldehyde-activated particles with an average diameter of 80 μm. The particles are washed in PBS (phosphate-buffered saline, 10 mM sodium phosphate, 150 mM NaCl, pH 7.4) by suspension and centrifugation and suspended in a ratio of 1:1 by volume. 5 μl of 20 μM amino-activated oligonucleotide dissolved in water are added to 50 μl of particle suspension. The particle suspension is incubated together with the oligonucleotide at room temperature with gentle shaking for one hour. It is possible to use as the oligonucleotide for example an oligonucleotide of the following sequence:

5′-Amino-TCCAAGCCTGGAGGGATGATACTTTGCGCTTGG-3′

A plastic template which has a cutout in the shape of the letter “A” is then placed on an amino-activated slide. The prepared particle suspension to which oligonucleotides have been added is dissolved in 10 mM NaOH by addition of 1 M sodium cyanoborohydride (supplied by Sigma, Munich) and adjusted to 50 mM sodium cyanoborohydride. The particle suspension is then applied to the template. The particle suspension comes into contact with the amino-activated surface of the slide through the cutouts in the template. The particle suspension has been incubated in contact with the surface of the slide in a humidity chamber at room temperature for about 20 hours.

Aldehyde-activated particles are then washed in PBS by suspension and centrifugation and suspended in a ratio of 1:1 by volume. 20 μl of 20 μM amino-activated other oligonucleotide dissolved in water are added to 2 μl of particle suspension. The particle suspension and the other oligonucleotide are incubated at room temperature with gentle shaking for one hour. An oligonucleotide of the following sequence has been used as other oligonucleotide:

5′-Amino-TTGGAATCCATGGTTAAACTTGTACTTTAGGTC-3′

The particles coated with the other oligonucleotide have been applied to the slide after removal of the template. Excess particles have been removed by aspiration with a glass capillary under the microscope. A plastic frame has been placed on the slide. The particle suspension with the other oligonucleotide has been brought to 50 mM sodium cyanoborohydride by addition of 1 M sodium cyanoborohydride dissolved in 10 mM NaOH. Particle suspension with other oligonucleotide has been applied inside the frame and incubated in a humidity chamber at room temperature overnight. To remove unbound particles, the slide has been washed several times in TE (10 mM TrisCl, 1 mM EDTA, pH 8) and stored in a humidity chamber in TE with 0.05% sodium azide.

To identify the mark produced by the oligonucleotide 1, a molecular beacon of the following sequence has been applied in a concentration of 50 nM dissolved in TE to the slide:

3′-X-GGTTCGGACCTCCCTACTATGAAACGCGAACC-6FAM-5′;

X=dt (C2-DABCYL).

The sequence of the molecular beacon is complementary to the sequence of the oligonucleotide. The solution has been applied by means of an atomizer to the slide at a temperature of 37° C. The slide has been irradiated before and after addition of the solution with light with a wavelength of 496 nm. The emission at a wavelength of 516 nm has been measured 5 minutes after addition of the solution.

FIG. 4 shows particles with oligonucleotide covalently bonded thereto on an amino-coated surface of a slide. The particles have been applied in the form of the letter “A”. They form a first part-pattern.

FIG. 5 shows the first part-pattern of FIG. 4 in combination with a second part-pattern. The second part-pattern is formed from particles which are coated with covalently bonded other oligonucleotide. The first and the second part-pattern cannot be distinguished from one another in transmitted light.

FIG. 6 shows the part-pattern of FIG. 5 after incubation with the molecular beacon which is complementary to the oligonucleotide. Owing to the hybridization of the molecular beacon with the oligonucleotide, a fluorescence can be observed on excitation with UV light after only a few minutes. The first part-pattern can be identified. It is distinctly evident in the form of the letter “A”.

In the second exemplary embodiment shown in FIG. 7, a polycarbonate sheet with a thickness of 0.25 mm was used as carrier. This was activated after cleaning with isopropanol in a first step by means of 5N NaOH for 30 min and then washed with H₂O.

Then, in a second step, the biopolymers were directly coupled to the carrier. Amino-modified DNA oligomers with a sequence N, which may be for example the previously described sequence, were used as biopolymer.

For this purpose, a solution of 5 μl of DNA oligomer (100 μM), 1 μl of 1-ethyl-3-(3-dimethylaminopropyl)-carbodiimide (EDC) and 4 pl of 0.1 M carbonate buffer, pH 9.5, was made up. In each case 1 μl of this solution was applied pointwise to the preactivated carrier so that a characteristic pattern resulted.

Binding was completed by incubation in a water-saturated atmosphere overnight and then excess DNA oligomers were removed by washing with H₂O and 0.1% Tween 20.

The binding was detected with another DNA oligomer in the form of a molecular beacon having the sequence N′, which was partly complementary to the sequence N. These were applied in a concentration of 1.0 pmol/μl to the mark and measured in a fluorescence microscope after about 30 s.

FIG. 7 shows a part-pattern from a DNA mark directly coupled to the carrier. One point from this mark is picked out separately in FIG. 8.

In a third exemplary embodiment, a polycarbonate sheet with a thickness of 0.25 mm was incubated with a mixture of 100 parts of ethanol and 1 part of glycidylsilane for 30 minutes. Silane is deposited on the surface during this. Excess silane was washed off the carrier with water, and the carrier was blown dry in a stream of nitrogen. The silane on the carrier was then crosslinked at 80° C. for 60 minutes. Amino-modified oligonucleotides with a sequence N, which may be for example the previously described sequence, are diluted in carbonate buffer; 0.1 M; pH 9.5; to concentrations of 0.5 pmol/μl, 1.0 pmol/μl and 2.0 pmol/μl and applied as drops 1 μl in size to the activated carrier and incubated for 30 minutes (FIGS. 9 to 11). The incubation lasted 6 hours with the sample shown in FIG. 12.

The carriers coated with DNA are incubated with a molecular beacon with the sequence N′, which is partly complementary to the sequence N, of concentration 1 pmol per μl for about 30 sec and then measured in a fluorescence microscope.

The result is evident from FIGS. 9 to 12:

a higher concentration of oligonucleotides in the drops increases the occupation density and leads to a brighter appearance of the mark produced. Thus, marks differing in intensity can also be produced by varying the concentration of the oligonucleotides.

List of Reference Numbers

10 area element,

14 first nucleic acid,

16 second nucleic acid,

20 molecular beacon,

22 fluorophore,

24 quencher,

26 loop,

30 solid body,

32 mark,

34 detector

36 output device

1. A method for identifying a predetermined mark (32) applied on a solid body (30) and formed from area elements (10), having the following steps:
a) binding of first biopolymers to a first part of the area elements (10) so that a first predetermined part-pattern is formed.
b) bringing the mark (32) into contact with third biopolymers having affinity for the first biopolymers, so that the first and the third biopolymers bind to one another and
c) identifying the first part-pattern formed by the bound first and third biopolymers through detecting the binding between the first and the third biopolymers by means of a one-stage detection method.

2. The method as claimed in claim 1, where the following step is carried out before step b: binding of second biopolymers to a second part of the area elements (10) so that a second part-pattern is formed.

3. The method as claimed in either of the preceding claims, where the biopolymers comprise, in particular synthetic and/or single-stranded, nucleic acids (14, 16), analogs thereof, antigens or proteins, in particular antibodies, antibody fragments, derivatives of an antibody or antibody fragment or nucleic acid-binding proteins.

4. The method as claimed in any of the preceding claims, where in step c) [sic] additionally fourth biopolymers having affinity for the second biopolymers are brought into contact with the mark (32), and where in step d) [sic] the bindings between the second and the fourth biopolymers are detected and the part-pattern formed by the bound second and fourth biopolymers is identified.

5. The method as claimed in any of the preceding claims, where the third and, where appropriate, the fourth biopolymers are present in a solution.

6. The method as claimed in any of the preceding claims, where the bringing into contact is carried out under predetermined stringent binding conditions, preferably at room temperature.

7. The method as claimed in any of the preceding claims, where at least one other or the second biopolymer is bound to the area elements (10) to saturate nonspecific binding sites.

8. The method as claimed in any of the preceding claims, where the first and second biopolymer are bound via hydrophilic linkers respectively to the one or other part of the area elements.

9. The method as claimed in any of the preceding claims, where the hydrophilic linkers are selected from the following group: peptides, polyethylene glycols, polymeric sugars, polyacrylamide, polyimines or dendrimer molecules.

10. The method as claimed in any of the preceding claims, where the hydrophilic linker is bound to the first or third biopolymer in a section which is not complementary respectively to the second or fourth biopolymer.

11. The method as claimed in any of the preceding claims, where at least one of the biopolymers is bound to the area elements (10) by means of particles, in particular agarose particles.

12. The method as claimed in any of the preceding claims, where at least one of the biopolymers is applied by means of a printing technique, in particular inkjet technique, to the area elements (10).

13. The method as claimed in any of the preceding claims, where the first and/or second biopolymers are bound at a predetermined site in their structure to the area elements (10).

14. The method as claimed in any of the preceding claims, where the part-pattern is in the form of a bar code.

15. The method as claimed in any of the preceding claims, where the part-pattern is designed in the form of an array.

16. The method as claimed in any of the preceding claims, where area elements (10) are designed to be round, preferably with a diameter of less than 100 μm.

17. The method as claimed in any of the preceding claims, where the binding is detected through altered optical and/or electrical properties of the bound biopolymers.

18. The method as claimed in any of the preceding claims, where at least one of the biopolymers has a fluorophore (22) which changes its fluorescence properties on binding.

19. The method as claimed in any of the preceding claims, where at least one of the biopolymers has a marking substance which changes the redox potential thereof on binding.

20. The method as claimed in any of the preceding claims, where the third and/or fourth biopolymers are brought into contact with the mark (32) homogeneously distributed by dropwise application, absorption, spraying or atomization.

21. The method as claimed in any of the preceding claims, where the one-stage detection method is carried out without washing steps.

22. The method as claimed in any of the preceding claims, where the one-stage detection method is carried out utilizing one of the following effects:
aa) formation or separation of a donor/acceptor pair,
bb) surface plasmon resonance,
cc) weight difference,
dd) inclusion or release of intercalators.

23. The method as claimed in any of the preceding claims, where the mark comprises the first biopolymer in an amount not exceeding 10 μg.

24. A carrier for attachment to a solid body, where a predetermined mark formed from area elements (10) is applied to one side of the carrier,
where first biopolymers are bound to a first part of the area elements (10) so that a first part-pattern is formed, and
where the carrier is designed as a sheet which is coated on the other side with adhesive.

25. The carrier as claimed in claim 24, where one side is covered with a detachable protective sheet.

26. The carrier as claimed in claim 24 or 25, where the adhesive layer is covered with another detachable protective sheet.

27. The carrier as claimed in any of claims 24 to 26, where second biopolymers are bound to a second part of the area elements (10) so that a second part-pattern is formed.

28. A kit comprising a carrier as claimed in any of claims 24 to 27 and comprising a third biopolymer having affinity for the first biopolymer.

29. The kit as claimed in claim 28, where a fourth biopolymer having affinity for the second biopolymer is present.
